(19) United States
(12) Patent Application Publication
(10) Pub. No.: US 2021/0386287 A1
(43) Pub. Date: Dec. 16, 2021

(54) DETERMINING REFRACTION USING ECCENTRICITY IN A VISION SCREENING SYSTEM

(71) Applicant: Welch Allyn, Inc., Skaneateles Falls, NY (US)

(72) Inventors: Yaolong Lou, Singapore (SG); See Heng Lee, Singapore (SG)

(73) Assignee: Welch Allyn, Inc.

(21) Appl. No.: 17/347,079

(22) Filed: Jun. 14, 2021

Related U.S. Application Data
(60) Provisional application No. 63/039,701, filed on Jun. 16, 2020.

(51) Int. Cl. A61B 3/103; A61B 3/00; A61B 3/11

(52) U.S. Cl. CPC A61B 3/103; A61B 3/112; A61B 3/0008

(57) ABSTRACT

A system for determining refractive error of an eye includes at least two light-emitting diodes (LEDs), an image capture component, and a processor configured to execute instructions to perform operations associated with a vision screening exam. For example, the processor causes each of the two LEDs to emit light to form a combined light that is output to a pupil. The processor causes the image capture component to capture an image depicting the eye while the combined light is being output. The processor determines an eccentricity at a time that the image was captured, corresponding to a simulated source location of the combined light caused by combining the light from the two LEDs. The processor determines an amount of light reflected on the pupil in the image, and a refractive error of the eye based on the eccentricity and the amount of light reflected on the pupil in the image.

### CROSS-REFERENCE TO RELATED APPLICATIONS

This application claims priority to U.S. Provisional Application No. 63/039,701, filed on Jun. 16, 2020, the entire contents of which are incorporated by reference herein.

### TECHNICAL FIELD

This application is directed to medical equipment, and in particular, to systems and methods associated with determining refractive error using an eccentricity measurement using a vision screening device.

### BACKGROUND

Visual screening in children and adults typically includes one or more tests to determine various deficiencies associated with the patient's eyes. Such vision tests may include, for example, refractive error tests, convergence tests, accommodation tests, visual acuity tests, and the like. Conventional vision tests may include the use of an ophthalmic testing device called a phoropter, which uses different lenses for refraction of the eye to measure an individual's refractive error, and in some cases may be used to determine an eyeglass prescription. Conventional phoropters rely upon a patient's feedback on various trial lenses, and in some cases this technique for relying upon feedback from the patient can lead to inaccurate results, such as with small children who may have difficulty communicating during an eye exam.

Objectively measuring neutralization of the eye may resolve the subjective nature of feedback (or difficulty in obtaining feedback from some patients such as small children). Neutralization refers to a state in the refraction test of an eye at which the eye shares an identity of a certain optical representation with an emmetropic eye. For example, a vision screening device can be used to direct light onto a person's retinas to measure neutralization. Sensors on the device may then collect corresponding light that is reflected by the retinas. The screening device determines that neutralization has been achieved when the light directed onto the person's retinas (or an amount of light below a threshold amount) is no longer detected by the sensors. The screening device may determine a refractive error of each eye based on characteristics of the reflected light, and/or characteristics of the light directed onto the person's retinas that achieved neutralization.

Controlling the output of light for determining neutralization in a vision screening device is not without challenges. For example, some conventional systems use mechanical movement of lenses and/or light sources (e.g., moving the lenses and/or light sources closer or further to a person) to determine when neutralization is achieved. However, mechanical movement of lenses and/or light sources often increases cost of the vision screening device and adds size to the vision screening device, making the vision screening device bulky and difficult to use in performing eye examinations that may require precise measurements. As a result, vision screening determinations made using existing devices may lack accuracy and consistency.

The various examples of the present disclosure are directed toward overcoming one or more of the deficiencies noted above.

### SUMMARY

In an example of the present disclosure, a system includes a first light-emitting diode (LED), a second LED, an image capture component, one or more processors, and one or more computer-readable media storing instructions that, when executed by the one or more processors, perform operations. For example, the operations may comprise causing the first LED to emit first light and the second LED to emit second light to form a combined light that is output to a pupil of an eye of a patient. The operations may further comprise causing the image capture component to capture an image depicting the eye while the combined light is being output to the pupil. In examples, the operations further comprise determining an eccentricity associated with the combined light at a time that the image was captured, where the eccentricity corresponds to a simulated source location of the combined light caused by combining the first light and the second light. The operations may further comprise determining an amount of light reflected on the pupil in the image. The operations may further comprise determining a refractive error of the eye based at least in part on the eccentricity and the amount of light reflected on the pupil in the image.

In another example of the present disclosure, a method includes causing a first LED of a vision screening device to emit first light and a second LED of the vision screening device to emit second light to form a combined light that is output to a pupil of an eye of a patient. Such a method also includes capturing, by an image capture component of the vision screening device, an image depicting an eye while the combined light is being output to the pupil. Such a method also includes determining an eccentricity associated with the combined light at a time that the image was captured, the eccentricity corresponding to a simulated source location of the combined light caused by combining the first light and the second light. Such a method also includes determining an amount of light reflected on the pupil in the image. Such a method further includes determining a refractive error of the eye based at least in part on the eccentricity and the amount of light reflected on the pupil in the image.

In another example of the present disclosure, one or more computer-readable media store instructions that, when executed by one or more processors, perform operations. For example, the operations may comprise causing a first light-emitting diode (LED) of a vision screening device to emit first light and a second LED of the vision screening device to emit second light to form a combined light that is output to a pupil of an eye of a patient. The operations may further comprise capturing, by an image capture component of the vision screening device, an image depicting the eye while the combined light is being output to the pupil. The operations may also comprise determining an eccentricity associated with the combined light at a time that the image was captured, where the eccentricity corresponds to a simulated source location of the combined light caused by combining the first light and the second light. The operations may also comprise determining an amount of light reflected on the pupil in the image. The operations may further comprise determining a refractive error of the eye based at least in part on the eccentricity and the amount of light reflected on the pupil in the image.

### BRIEF DESCRIPTION OF THE DRAWINGS

Features of the present disclosure, its nature, and various advantages, may be more apparent upon consideration of the following detailed description, taken in conjunction with the accompanying drawings.

FIG. 1A illustrates an isometric view of an example system of the present disclosure. In some implementations, components of the example system shown in FIG. 1 may be used to perform one or more tests associated with vision screening.

FIG. 1B illustrates a sectional view of the example system of the present disclosure.

FIG. 2 illustrates components of a vision screening device according to examples of the present disclosure.

FIG. 3 illustrates example characteristics that may be used by a vision screening device to determine features of a pupil from an image, according to examples of the present disclosure.

FIG. 4 provides a schematic illustration of current values that may be applied by a vision screening device to LEDs to generate a combined light position according to examples of the present disclosure.

FIG. 5 provides a first portion of a first flow diagram illustrating an example method of the present disclosure.

FIG. 6 provides a second portion of a first flow diagram illustrating an example method of the present disclosure.

FIG. 7 provides a second flow diagram illustrating an example method of the present disclosure.

FIG. 8 provides a schematic illustration of an example vision screening device of the present disclosure.

In the figures, the left-most digit(s) of a reference number identifies the figure in which the reference number first appears. The use of the same reference numbers in different figures indicates similar or identical items or features. The drawings are not to scale.

### DETAILED DESCRIPTION

The present disclosure is directed to, in part, a vision screening system and corresponding methods. Such an example vision screening system may be configured to perform one or more vision tests on a patient and to output the results of the vision test(s) to a user of the device, such as a physician or a physician's assistant. For example, the vision screening system may generate one or more graphical representations, such as a series of characters (e.g., a Snellen chart), images, or other items useful for testing the visual acuity of the patient. The system may also generate one or more beams of radiation, and may be configured to direct such beams at the retinas of the patient. The system may collect corresponding light that is reflected back from the retinas, and may determine a refractive error of the patient's eyes based at least in part on characteristics of the collected light.

In some examples, the system determines the refractive error based on eccentricity of one or more beams relative to a pupil of a person's eye that achieved neutralization of the beams. Eccentricity refers to a distance in a perpendicular direction from a line that connects a center of a patient's pupil and a center of a camera capturing an image of the pupil in a vision screening device to a position of a light source used to illuminate the pupil. By moving the beams of light, the system determines an eccentricity (e.g., perpendicular distance from the line between the pupil and the camera center) at which neutralization is achieved, i.e., when the light reflection moves just out of the pupil. The system may then determine refractive error based, in part, on the eccentricity at which neutralization was achieved. In some examples, rather than mechanically moving the light sources used to determine refractive error, the system may adjust the intensities of the light sources to simulate movement. As such, in any of the examples described herein, the results of the various vision tests performed using the system may include one or more measurements obtained by the vision screening device included in the system. In addition, the system may generate a recommendation and/or diagnosis associated with the patient for display to the user of the vision screening device. For example, by utilizing standard testing data and/or machine learning techniques, the system may evaluate the measurements determined by the system to provide a recommendation to the user regarding the vision of the patient (e.g., whether the patient passed the test, whether the patient has myopia and/or hyperopia, whether the patient requires additional screening, etc.). As such, the system described herein may provide automated diagnosis recommendations in order to assist the physician or other user of the vision screening device.

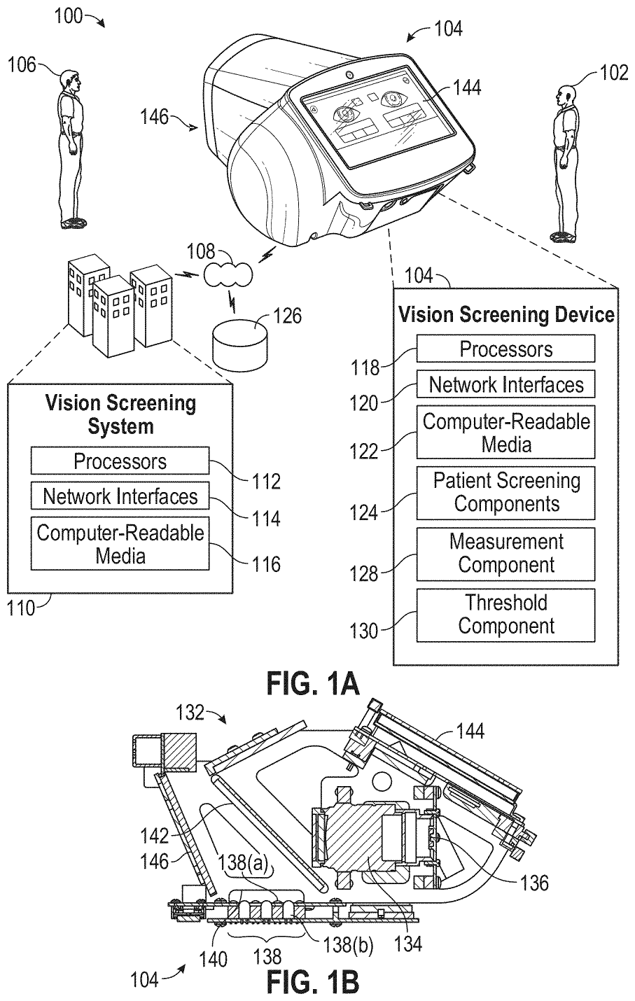

Additional details pertaining to the above-mentioned techniques are described below with reference to FIGS. 1-8. It is to be appreciated that while these figures describe example systems and devices that may utilize the claimed methods, the methods, processes, functions, operations, and/or techniques described herein may apply equally to other devices, systems, and the like.

FIG. 1A illustrates an example system 100 for vision screening according to some implementations. As illustrated in FIG. 1A, a user 102 may utilize a vision screening device 104 and/or other components of the system 100 to administer a vision screening test on a patient 106 to determine the vision health of the patient 106. FIG. 1B illustrates a cross-sectional view of an example vision screening device 104. As described herein, the vision screening device 104 may perform one or more vision screening tests to determine one or more measurements associated with the patient 106, and may provide the measurement(s), via a network 108, to a local or remote vision screening system 110 for analysis. In response, the vision screening system 110 may analyze the measurement(s) to diagnose the vision health of the patient 106. It should be understood that, while FIG. 1A depicts the system 100 including a single vision screening system 110, in additional examples, the system 100 may include any number of local or remote vision screening systems substantially similar to the vision screening system 110, and configured to operate independently and/or in combination, and configured to communicate via the network 108. In examples, the vision screening system 110 may include one or more processors 112, one or more network interfaces 114, and/or computer-readable media 116. The computer-readable media 116 may store one or more programs, modules, engines, instructions, algorithms, and so forth that are executable by the processor(s) 112. Similarly, the vision screening device 104 may include one or more processors 118, one or more network interfaces 120, and/or computer-readable media 122. The computer-readable media 122 may store one or more programs, modules, engines, instructions, algorithms, and/or other patient screening components 124 that are executable by the processor(s) 112.

In examples, the vision screening device 104 may include a stationary or portable device configured to perform one or more vision screening tests on the patient 106. For example, the vision screening device 104 may be configured to perform a visual acuity test, a refractive error test, an accommodation test, dynamic eye tracking tests, and/or any other vision screening tests configured to evaluate and/or diagnose the vision health of the patient 106. Due to its stationary or portable nature, the vision screening device 104 may perform the vision screening tests at any location, from conventional screening environments, such as schools and medical clinics, to physician's offices, hospitals, eye care facilities, and/or other remote and/or mobile locations.

As described herein, the vision screening device 104 and/or vision screening system 110 may be configured to perform accommodation and refractive error testing on the patient 106. For example, refractive error and accommodation testing may include displaying a visual stimulus, such as a light or graphical representation, configured to induce a strain to the patient 106's eyes. In response, the vision screening device 104 may detect the pupils and/or lenses of the eyes of the patient 106, acquire images and/or video data of the pupils/lenses, and the like, and may transmit the vision screening data, via the network 108, to the vision screening system 110 for analysis. Alternatively, or in addition, the vision screening device 104 may perform the analysis locally.

In examples, the vision screening device 104 may also be configured to perform visual acuity testing and/or dynamic eye tracking tests. For example, the vision screening device 104 and/or the vision screening system 110 may be configured to perform visual acuity testing, which includes determining an optotype, determining a distance of the patient 106 from the vision screening device 104, and/or displaying a static or dynamic optotype to the patient 106. The dynamic eye tracking test may include generating a graphical representation, such as a graphic scene or text, for display to the patient 106 and monitoring the movement of the eye, acquire images and/or video data of the eyes, and the like, and may transmit the vision screening data, via the network 108, to the vision screening system 110 for analysis. Alternatively, or in addition, in some examples, the vision screening device 104 may analyze the vision screening data locally.

In examples, a memory associated with the vision screening device 104 and/or one or more of the patient screening components 124 may be configured to store and/or access data associated with the patient 106. For example, the patient 106 may provide data (referred to herein as “patient data”) upon initiating a vision screening test. For instance, when the vision screening device 104 and/or vision screening system 110 initiates a vision screening test, the patient 106 may provide, or the user 102 may request, patient data including the patient's demographic information, physical characteristics, preferences, and the like. For example, the patient 106 may provide demographic information such as name, age, ethnicity, gender, and the like. The patient 106 may also provide physical characteristic information such as height of the patient 106. In such examples, the user 102 may request the patient data while the screening is in progress, or before the screening has begun. In some examples, the user 102 may be provided with predetermined categories associated with the patient 106, such as predetermined age ranges (e.g., six to twelve months, one to five years old, etc.), and may request the patient data in order to select the appropriate category associated with the patient 106. In other examples, the user 102 may provide a free form input associated with the patient data. In still further examples, an input element may be provided to the patient 106 directly.

The vision screening device 104 may be configured to generate image and/or video data associated with the patient 106 at the onset of the vision screening test. For example, the vision screening device 104 may include one or more digital cameras, motion sensors, proximity sensors, infrared sensors, or other image capture devices configured to collect images and/or video of the patient 106, and one or more processors of the vision screening device 104 may analyze the collected images and/or video to determine, for example, the height of the patient 106, the distance of the patient 106 from the screening device, and/or any of the patient data described above.

Alternatively, or in addition, the vision screening device 104 may be configured to transmit the images, video, and/or any other collected information to the vision screening system 110, via the network 108, for analysis. In any such examples, the vision screening system may store such information in the computer-readable media 116, the computer-readable media 122, and/or in an external database 126. For example, the database 126 may comprise memory or other computer-readable media substantially similar to and/or the same as the computer-readable media 116 or the computer-readable media 122. The database 126 may be accessible by the vision screening system 110, and/or by the vision screening device 104, via the network 108. In any such examples, the database 126 may be configured to store patient data in association with a patient ID (e.g., a name, social security number, an alphanumeric code, etc.) or other unique patient identifier. When the user 102 and/or patient 106 enters the patient ID, the patient screening component 124 may access or receive patient data stored in association with the patient ID.

In some examples, the computer-readable media 122 may additionally store a measurement component 128. In such examples, the measurement component 128 may be configured to receive, access, and/or analyze testing data collected and/or detected by the vision screening device 104 during one or more vision screening procedures. For example, the measurement component 128 may be configured to receive, via one or more sensors of the vision screening device 104, image data and/or video data generated by the sensors during a vision screening test, such as while a graphical representation (e.g., a Snellen chart, a dynamic visual stimulus, etc.) is being displayed by the vision screening device 104. The measurement component 128 may analyze the image data and/or video data to determine one or more measurements associated with the patient 106, such as the gaze of the patient throughout the screening, a location of the patient's pupils at points in time of viewing the graphical representation, a diameter of the pupils, an accommodation of the lens, motion information associated with the eyes of the patient 106, and the like. In some cases, the measurement component 128 may determine a refractive error of one or both eyes of the patient 106 using an eccentricity measurement. As described in more detail below, the measurement component 128 may determine whether, and how much, light reflects from one or both pupils of the patient 106 when the vision screening device 104 directs light toward the pupils. The measurement component 128 may use this information to determine an angle of the light directed at the one or more pupils of the patient 106 at which neutralization is achieved, and then may determine a refractive error based on the angle of neutralization.

Further, the computer-readable media 122 may also be configured to store a threshold data component 130. The threshold data component 130 may be configured to receive, access, and/or analyze threshold data associated with standard vision testing results. For example, the threshold data component 130 may be configured to access or receive data from one or more additional databases (e.g., the database 126, a third-party database, etc.) storing testing data, measurements, and/or a range of values indicating various thresholds or ranges within which testing values should lie. Such thresholds or ranges may be associated with patients having normal vision health with similar testing conditions. For example, for each testing category, standard testing data may be accessed or received by the threshold data component 130, and may be utilized for comparison against the measurement data stored by the measurement component 128 described above. For instance, the threshold data associated with the toddler testing category may include standard pupil measurements, and/or a threshold range of values which the testing values should not exceed or fall below (e.g., a standard value range) for toddlers when displayed each graphical representation. For example, when testing for accommodation in the patient 106, the threshold data component 130 may be configured to store information associated with the amplitude of accommodation and age (e.g., Donder's Table).

As used herein, the network 108 is typically any type of wireless network or other communication network known in the art. Examples of network 108 include the Internet, an intranet, a wide area network (WAN), a local area network (LAN), and a virtual private network (VPN), cellular network connections and connections made using protocols such as 802.11a, b, g, n and/or ac. U.S. Pat. No. 9,237,846, granted on Jan. 19, 2016, describes systems and methods for photo refraction ocular screening and that disclosure is hereby incorporated by reference in its entirety.

As described herein, a processor, such as processor(s) 112 and/or 118, can be a single processing unit or a number of processing units, and can include single or multiple computing units or multiple processing cores. The processor(s) 112 and/or 118 can be implemented as one or more microprocessors, microcomputers, microcontrollers, digital signal processors, central processing units, state machines, logic circuitries, and/or any devices that manipulate signals based on operational instructions. For example, the processor(s) 112 and/or 118 can be one or more hardware processors and/or logic circuits of any suitable type specifically programmed or configured to execute the algorithms and processes described herein. The processor(s) 112 and/or 118 can be configured to fetch and execute computer-readable instructions stored in the computer-readable media 116 and/or 122, which can program the processor(s) 112 and/or 118 to perform the functions described herein.

The computer-readable media 116 and/or 122 may can include volatile and nonvolatile memory and/or removable and non-removable media implemented in any type of technology for storage of information, such as computer-readable instructions, data structures, program modules, or other data. Such computer-readable media 116 and/or 122 can include, but is not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. The computer-readable media 116 and/or 122 can be a type of computer-readable storage media and/or can be a tangible non-transitory media to the extent that when mentioned, non-transitory computer-readable media exclude media such as energy, carrier signals, electromagnetic waves, and signals per se.

The computer-readable media 116 and/or 122 can be used to store any number of functional components that are executable by the processor(s) 112 and/or 118. In many implementations, these functional components comprise instructions or programs that are executable by the processor(s) 112 and/or 118 and that, when executed, specifically configure the one or more processor(s) 112 and/or 118 to perform the actions associated with one or more vision tests.

The network interface(s) 114 and/or 120 may enable wired and/or wireless communications between the components and/or devices shown in system 100 and/or with one or more other remote systems, as well as other networked devices. For instance, at least some of the network interface(s) 114 and/or 120 may include a personal area network component to enable communications over one or more short-range wireless communication channels. Furthermore, at least some of the network interface(s) 114 and/or 120 may include a wide area network component to enable communication over a wide area network. Such network interface(s) 114 and/or 120 may enable, for example, communication between the vision screening system 110 and the vision screening device 104 and/or other components of the system 100, via the network 108.

Referring to FIG. 1B, a cross-sectional view 132 illustrates optical and non-optical components of the vision screening device 104. Optical components of the vision screening device 104 may include, for example, a lens component 134 coupled to an image capture component 136, a light-emitting diode (LED) array 138 having visible LEDs 138(a) and near-infrared LEDs 138(b), a diffuser 140, and a beam splitter 142. Non-optical components of the vision screening device 104 may include, for example, an operator display screen 144 and a patient view window 146. It is noted that the vision screening device 104 is not limited to the components listed here, and may incorporate additional components for furthering vision screening techniques.

In some examples, the vision screening device 104 may present attention-getting stimuli to the patient 106 to attract a direction of the gaze of the patient 106, such as for patients who may not readily cooperate with the vision screening exam (e.g., small children). In some cases, a gaze of the patient 106 being in a direction of the lens component 134 results in more accurate vision screening measurements. The attention-getting stimuli may be an auditory stimulus, such as a digitally recorded sound track under control of the processor 118 and may be presented by a speaker (not pictured) of the vision screening device 104. Alternatively or additionally, the attention-getting stimuli may be a visual stimulus presented by one or more of the visible LEDs 138(a) of the LED array 138.

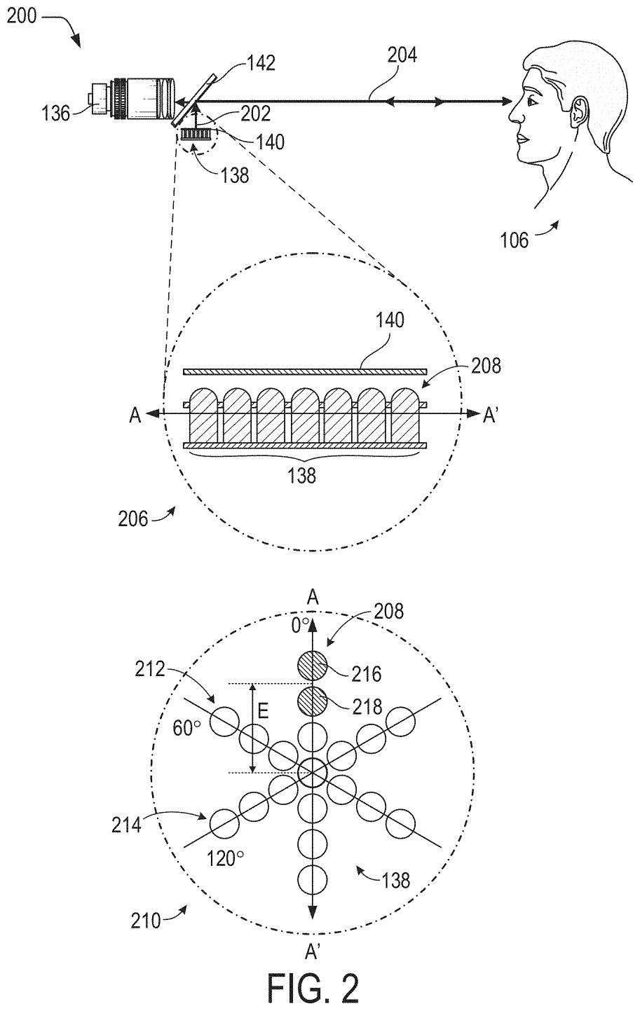

The visual stimulus may comprise an arrangement of differently-colored LEDs that emit visible light below a threshold wavelength (e.g., 600 nanometers) to avoid contamination of near infrared (NIR) LED stimulus. In examples, the NIR LEDs are used to capture pupil images to conduct ocular examination of the eye(s) of the patient 106. The NIR LEDs in the LED array may emit light at a wavelength of approximately 850 nanometers (nm) and may be configured to capture video and/or image data associated with the eye(s) of the patient 106. The configuration of the LED array 138 with differently-colored LEDs emitting light at a first wavelength and NIR LEDs emitting light at a second wavelength allows the visual stimulus to be presented for attention-getting purposes, without the visual stimulus being recorded in ocular examination images of the patient 106. Accordingly, the visible LED stimulus is independent of the NIR LED stimulus, and the visible LED stimulus may not be used in determining refractive error or gaze direction. The LED array 138 may be arranged with visible LEDs 138(a) positioned between and coplanar with NIR LEDs 138(b). In some examples, light emitted by the visible LEDs 138(a) passes through the diffuser 140, creating diffuse stimuli. After passing through the diffuser 140, the diffuse stimuli may be reflected towards the user 102 via the beam splitter 142. Additional details regarding the emission of light by the visible LEDs 138(a) and control of the emitted light by the diffuser can be found in the discussion of FIG. 2.

Similar to the auditory stimulus, the visible LEDs 138(a) may be under control of the processor 118 according to control parameters stored in the computer-readable media 122. For instance, control parameters may include intensity, duration, pattern, cycle time, and so forth for the visible LEDs 138(a). With respect to intensity, the control parameters may direct the visible LEDs 138(a) to emit light that is bright enough to attract attention of the patient 106, while also limiting brightness to avoid pupil constriction. The measurement component 128 may also use the control parameters to determine a duration that the visible LEDs 138(a) emit light (e.g., 50 milliseconds, 100 milliseconds, 200 milliseconds, etc.). Further, the measurement component 128 may use the control parameters to display patterns to the patient 106 using the visible LEDs, such as circular patterns, alternating light patterns, flashing patterns, patterns of different colors, shapes such as circles or rectangles, and the like.

In an illustrative example, the measurement component 128 may use the control parameters to present diffuse, random, and rapidly changing visible light patterns to the patient 106 using the visible LEDs 138(a). In examples, such patterns may reduce, and in some cases may inhibit, accommodation of the eye(s) of the patient 106 at a set focal distance (e.g., 1 meter from the image capture component 136 of the vision screening device 104).

In some examples, the measurement component 128 may use digital image feature detection and filtering for pupil location determinations in images captured by the image capture component 136. For instance, light emitted from the LED array 138 is reflected by the lens component 134 and transmitted along an optical axis towards the eye(s) of the patient 106, through the patient view window 146. Light reflects off of the eye(s) of the patient 106, and returns through the patient view window 146 to be received by the image capture component 136 of the vision screening device 104. The image capture component 136 may receive raw sensor data from an image sensor (not pictured) of the image capture component 136, may contrast-enhance the raw sensor data, and may transform the raw sensor data into a display format for presentation and feedback to the user 102 via the operator display screen 144. The image capture component 136 may cause the operator display screen 144 to present a display image relating to the raw sensor data. The measurement component 128 may cause additional information to be displayed on the operator display screen 144 as well, such as a distance of the patient 106 from the vision screening device 104, quality of focus, progress of the examination, and/or other information related to the vision screening examination process. The operator display screen 144 may comprise, for example, a liquid crystal display (LCD) or active matrix organic light emitting display (AMOLED). The operator display screen 144 may also be touch-sensitive to receive input from the user 102.

In examples, the measurement component 128 determines a refractive error of the eye(s) of the patient 106 based at least in part on an eccentricity of light output by two (or more) of the LEDs in the LED array 138, and an amount of light reflected on the pupil of the patient 106 as captured in an image by the image capture component 136. For instance, by applying current to two NIR LEDs 138(b), light emitted by the two NIR LEDs may combine and appear to the patient 106 to originate from a single source within the vision screening device 104. The diffuser 140 may cause the light emitted by the two NIR LEDs 138(b) to be output to the patient 106 at a desired combined intensity, as discussed in more detail below in relation to FIG. 2. The measurement component 128 may alter the amount of current applied to the two NIR LEDs 138(b) to simulate the source location of the combined light moving (e.g., radially out from a center of the LED array 138). In this way, although the combined light appears to the patient 106 to be moving, the vision screening device 104 does not require mechanical components to physically move the LED array 138, or individual LEDs within the LED array. Therefore, a manufacturing cost of the vision screening device 104 may be reduced by omitting mechanical components to determine refractive error based on eccentricity. Additionally, in some cases, the vision screening device 104 may take on a smaller form factor without such mechanical components to determine refractive error based on eccentricity, thus making the vision screening device 104 more portable for the user 102 to execute multiple vision screening exams on different patients.

FIG. 2 illustrates an example system 200 including components of the vision screening device 104 according to examples of the present disclosure. The example system 200 illustrates the patient 106, the image capture component 136, the LED array 138, the diffuser 140, and the beam splitter 142 of FIG. 1. Other components of the vision screening device 104 have been omitted in the example system 200 for clarity.

As discussed above, the measurement component 128 may instruct one or more LEDs in the LED array 138 to emit light 202. In examples, the light 202 passes through the diffuser 140 and strikes the beam splitter 142. At least a portion 204 of the light 202 reflects off of the beam splitter 142 and is directed to one or more eyes of the patient 106. While the portion 204 of the light 202 is directed at the eye(s) of the patient 106, the image capture component 136 captures an image of the eye(s) of the patient 106. In examples, the image may depict light that is reflected on the pupil(s) of the eye(s) of the patient 106 due to myopia and/or hyperopia, based on an angle of the portion 204 of the light 202 relative to the pupil and the condition of the myopia and/or hyperopia of the eye.

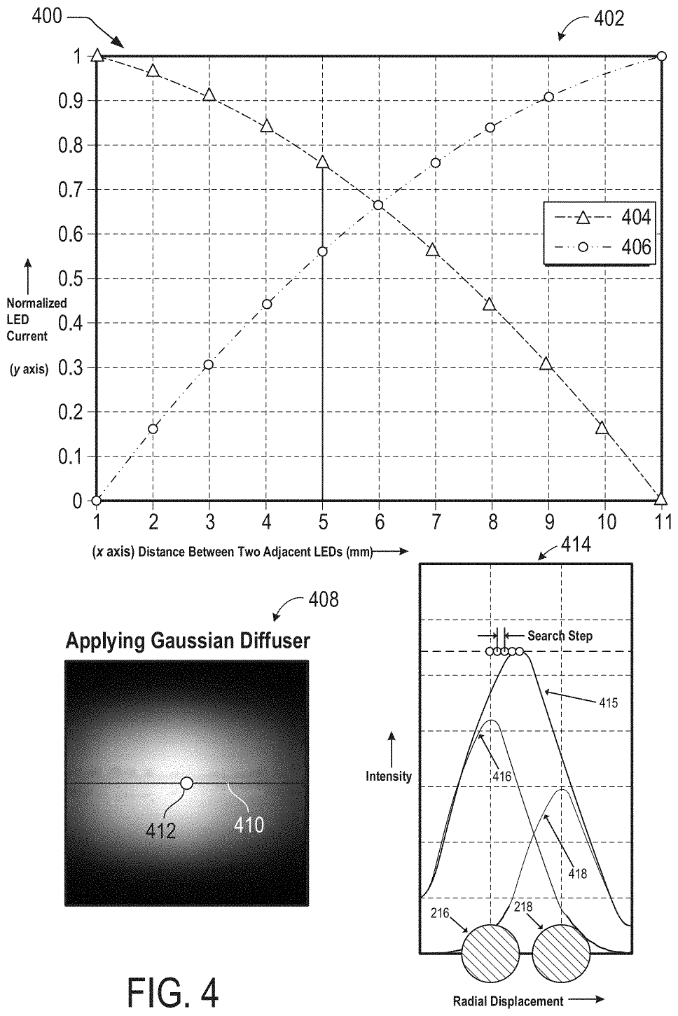

An expanded view 206 illustrates the LED array 138 and the diffuser 140 of the vision screening device 104. The expanded view 206 depicts a row 208 of LEDs in the LED array 138, that may extend generally in a line across the LED array 138 from A to A′. As discussed in greater detail in relation to FIG. 4, the diffuser 140 may act as a blur smooth filter for light emitted by the LEDs in the LED array 138. Therefore, in some cases, when two or more LEDs in the LED array emit light, the diffuser 140 may diffuse the emitted light 202 such that the portion 204 of the light appears to the patient 106 to originate from a single source.

A plan view 210 illustrates an example orientation of the LED array 138 from an orientation above the LED array 138. In some cases, the LED array 138 may include more or fewer individual LEDs than those shown in the example orientation. The plan view 210 includes the row 208 of LEDs illustrated in the expanded view 206, that extend generally in the line across the LED array 138 from A to A′, forming a meridian of the LED array 138. In examples, the row 208 forming the meridian from A to A′ may be a first meridian of the LED array 138, and the LED array 138 may include a row 212 at a meridian at 60 degrees relative to the row 208 and a row 214 at 120 degrees relative to the row 208.

As discussed above, the measurement component 128 of the vision screening device 104 may cause two (or more) LEDs of the LED array 138 to emit light substantially simultaneously in order to simulate a source location of the combined light with help of the diffuser 140. In the example shown, an LED 216 and an LED 218 are hatched to represent these two LEDs emitting light. The measurement component 128 may control an amount of current applied to the LED 216 and the LED 218 to achieve a desired simulated source location of the combined light at an eccentricity “E” from a center of the LED array 138. By varying an amount of current applied to the LED 216 and/or applied to the LED 218, the measurement component 128 can change the eccentricity, or simulated source location of the combined light, without having to mechanically move the LED array 138.

For instance, the measurement component 128 may alter an amount of current applied to every two adjacent LEDs like LED 216 and/or applied to the LED 218 such that the simulated source location travels from the center of the LED array 138 along the meridian formed by the row 208 to the exterior of the LED array 138. The measurement component 128 may continue altering the current applied to the LED 216 and/or applied to the LED 218 (or other LEDs in the row 208) until measurement component 128 determines that neutralization of the eye(s) of the patient 106 has been achieved. The measurement component 128 may then move to the row 212 and/or the row 214 and perform a similar process of applying varying amounts of current to two or more LEDs in the row to generate simulated source location(s) along the respective meridians until neutralization is achieved along each meridian. The measurement component 128 may then determine a refractive error of the eye(s) of the patient based on the eccentricity that achieved neutralization along each of the meridians formed by the rows 208, 212, and/or 214.

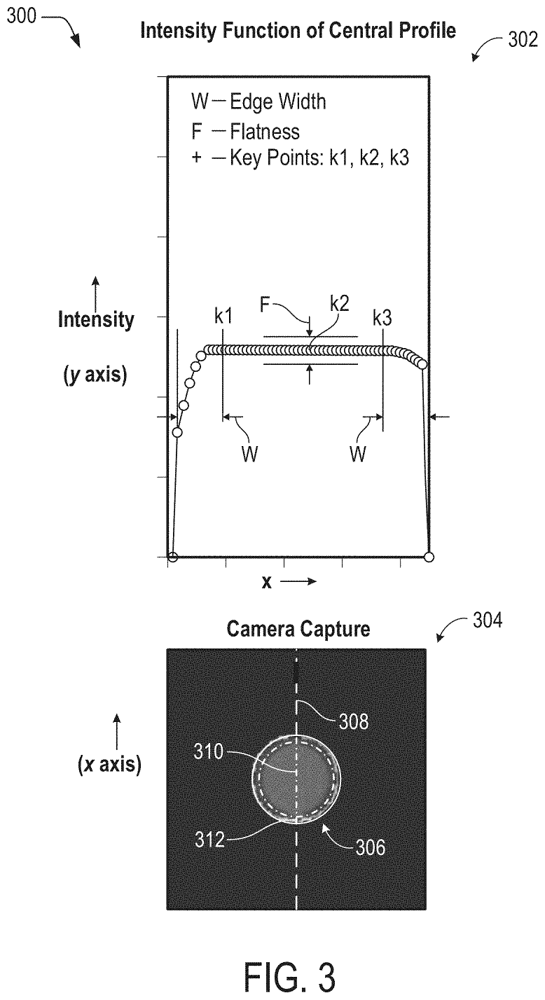

FIG. 3 illustrates an example 300 of characteristics that may be used by a vision screening device to determine features of a pupil from an image, according to examples of the present disclosure. In some examples, the measurement component 128 may determine characteristics of an eye of a patient prior to determining a target eccentricity that neutralizes refractive error of the eye. For instance, the measurement component 128 may determine a diameter of the pupil of the eye, locations of edges of the pupil, and the like, which may be used throughout the process of determining the refractive error. The measurement component 128 may use one or more images captured by the image capture component 136 to determine the described characteristics.

For example, the measurement component 128 may determine an intensity function of a central profile of a pupil represented in an image captured by the image capture component 136, as shown in a chart 302. The chart 302 illustrates a location along an axis of the image (the x axis of the chart 302) and an intensity value of a pixel found at the location in the image (they axis of the chart 302). The measurement component 128 may determine an edge width “W” of the pupil based on a slope between two or more intensity values being greater than a threshold amount (e.g., greater than 0.5, greater than 1, greater than 2, etc.). Additionally, in some examples, the measurement component 128 may determine a flatness “F” corresponding to the area of the pupil inside of the edge based on a slope between two or more intensity values being less than a threshold amount (e.g., less than 1, less than 0.5, less than 0.25, etc.).

Additionally, in some examples, the measurement component 128 may determine key points k1, k2, and k3, which represent a meridian central profile of the pupil. For instance, the key point k1 may correspond to a first interior of the edge of the pupil, the key point k2 may correspond to a location of the center of the flatness area “F” of the pupil, and the key point k3 may correspond to a second interior edge of the pupil opposite the first interior edge at k1. In examples, the measurement component 128 may compare intensity values at these locations to one another to determine whether the eye is emmetropic, myopic, or hyperopic at this meridian.

For example, during a vision screening exam, if the measurement component 128 determines that the difference between the value of the intensity at k1 and the value of the intensity at k3 is less than a threshold intensity and the intensity value at the location of k2 is less than a threshold amount, the measurement component 128 may determine that the eye is emmetropic at this meridian. If the measurement component 128 determines that the difference in intensity values at the key points k1 and k3 are greater than the threshold intensity, the measurement component 128 may begin applying current to LEDs in the LED array 138 along a meridian as described herein to determine an eccentricity that minimizes the light reflected on the pupil. For instance, the measurement component 128 may apply current to the LED 216 and the LED 218 to simulate a source location of the combined light from both of the LEDs 216, 218, where the simulated source location is based on a diameter of the pupil determined from the intensity function of the central profile.

In some examples, the measurement component 128 may cause, in a first step of an eccentricity determination, the simulated source location to appear at 0.25*d from the pupil center at k2, where d corresponds to the diameter of the pupil of the patient 106. The measurement component 128 may instruct the image capture component 136 to capture an image of the pupil while the pupil is illuminated by the combined light at the simulated source location. Then, the measurement component 128 may compare an intensity value at k1 to an intensity value at k3 to determine whether the eye is myopic or hyperopic at this meridian. For instance, if the intensity value at k1 is greater than the intensity value at k3, the measurement component 128 may determine that the eye is myopic at this meridian. Otherwise, if the intensity value at k1 is less than the intensity value at k3, the measurement component 128 may determine that the eye is hyperopic at this meridian. The measurement component 128 can use this determination to alter the simulated source location of the combined light in a direction of the greater intensity value (k1 or k3) along the meridian until neutralization is achieved by the intensity values at k1 and k3 are within the threshold amount mentioned above.

With continued reference to FIG. 3, an image 304 illustrates a camera capture of a pupil 306 at which neutralization has been achieved using the techniques described herein. The x axis and they axis represent locations in the image 304, such as pixel values within the image 304. A dashed line 308 extends vertically across the image 304, which may correspond to the line used by the measurement component 128 to generate the central profile described above. For instance, the values on the x axis of the chart 302 may correspond to the locations along the line 308 in the image 304. Additionally, the intensity values in the chart 302 may correspond to intensities of pixels at locations along the line 308 in the image 304. A portion 310 of the line 308 may correspond to the location of the pupil in the image 304, and in some cases, an edge width 312 (as determined by a slope between two or more intensity values, as described above) may be omitted from eccentricity analysis.

FIG. 4 provides a schematic illustration 400 of current values that may be applied by a vision screening device to LEDs to generate a combined light position according to examples of the present disclosure. A chart 402 illustrates normalized currents on the y axis that the measurement component 128 may apply to the LEDs in the LED array 138, including (but not limited to) the LEDs 216 and 218 based on the combined light spot position between the two LEDs on the x-axis. The functions illustrated in the chart 402 for a current 404 applied to a first LED and a current 406 applied to a second LED may be used to control a simulated source location of combined light between the two LEDs, as described herein. For instance, the measurement component 128 may apply current according to the function for the current 404 to the LED 216, and apply current according to the function for the current 406 to the LED 218, to simulate movement of the combined light of the LED 216 and the LED 218. In some cases, as shown in the chart 402, the measurement component 128 may use non-linear functions to control the current 404 and/or the current 406.

In some examples, the diffuser 140 may cause the combined light from the LED 216 and the LED 218 to appear from a single source as the simulated source location moves due to the change in intensity of the two LEDs. For instance, an image 408 illustrates an appearance of combined light output by the LED 216 and the LED 218 with the diffuser 140 in place to diffuse the combined light output by the two LEDs. The x axis and they axis include values that may represent locations in the image 408, such as based on pixel values of the image 408. As discussed above, the diffuser 140 may act as a blur smooth filter. Additionally, in some examples, the diffuser 140 can model as a Gaussian blur smooth filter that allows intensities of light to pass through based on a full-width at half-maximum on a Gaussian curve of intensities output by the LEDs. A line 410 overlaying the image 408 may represent a meridian along which a row (e.g., the row 208) of the LED array 138 is located, and along which the measurement component 128 performs an eccentricity analysis. A circle 412 overlaying the image 408 may represent a simulated source location of the combined light of two or more LEDs in the LED array 138, such as the LED 216 and the LED 218 described in FIG. 2.

As illustrated in FIG. 4, a chart 414 shows the intensity 415 of combined light spot 412 generated by LED 216 and LED 218 when their currents 404 and 406 produce the intensity 416 and intensity 418 respectively. The small circles are the search steps where the search starts from the center of one LED towards the center of the adjacent one.

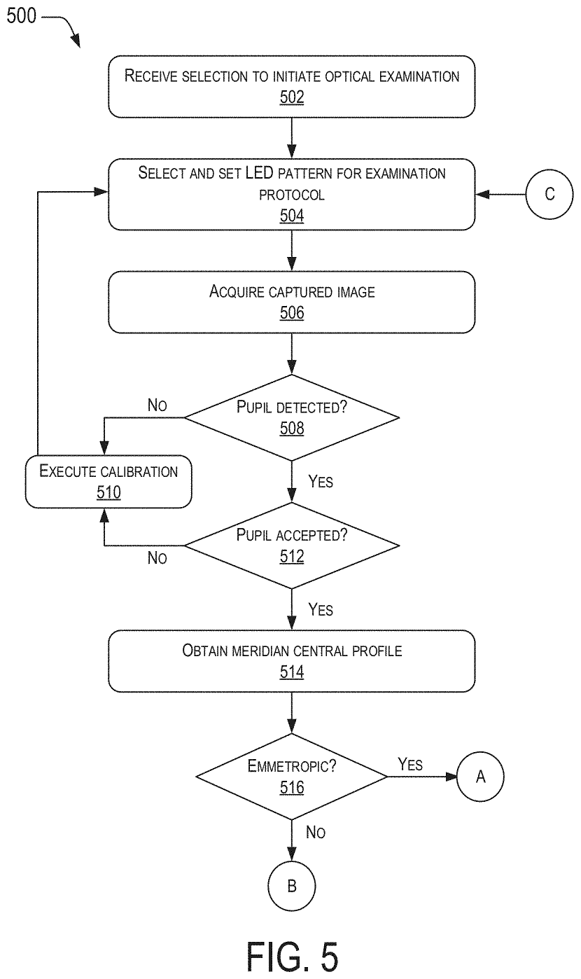

FIGS. 5-7 provide flow diagrams illustrating example methods for vision screening, as described herein. The methods in FIGS. 5-7 are illustrated as collections of blocks in a logical flow graph, which represents sequences of operations that can be implemented in hardware, software, or a combination thereof. In the context of software, the blocks represent computer-executable instructions stored on one or more computer-readable storage media that, when executed by processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the methods illustrated in FIGS. 5-7. In some embodiments, one or more blocks of the methods illustrated in FIGS. 5-7 can be omitted entirely.

The operations described below with respect to the methods illustrated in FIGS. 5-7 can be performed by any of the systems 100, 200, and 800 described herein, and/or by various components thereof. Unless otherwise specified, and for ease of description, the methods illustrated in FIGS. 5-7 will be described below with reference to the system 100 shown in FIG. 1. In particular, although any of the operations described with respect to the methods illustrated in FIGS. 5-7 may be performed by the measurement component 128 of the vision screening device 104, the threshold component 130, the one or more processors 112 of the vision screening system 110, the one or more processors 118 of the vision screening device 104, and/or other components of the system 100, either alone or in combination, the method 300 will be described below with respect to the system 100 and/or the measurement component 128 unless otherwise specified.

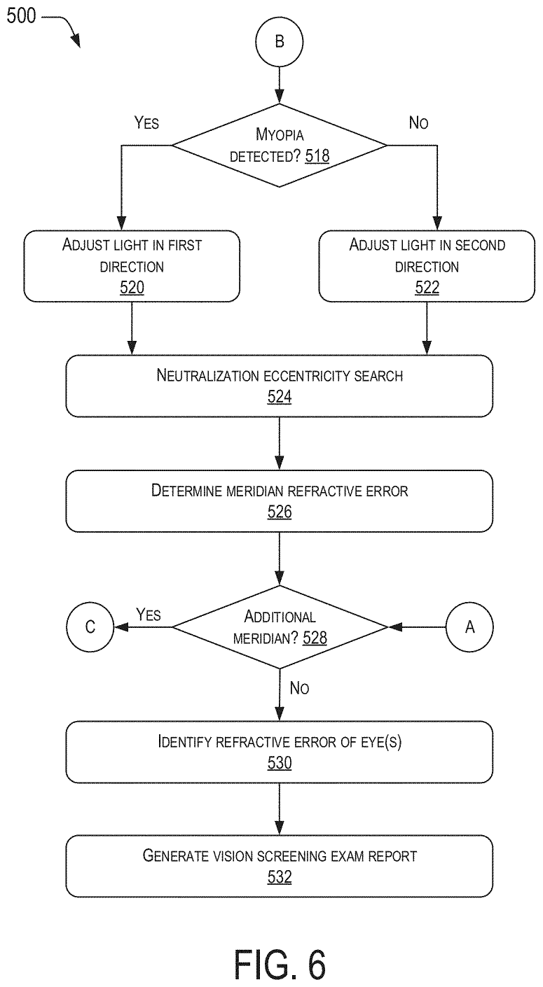

FIGS. 5 and 6 provide a first flow diagram illustrating an example method 500 of the present disclosure. At 502, the measurement component 128 and/or one or more processors associated therewith receive a selection to initiate an optical examination. For example, the measurement component 128 may receive an indication of a selection from a touch interface included as part of the operator display screen 144 and/or one or more other controls of the vision screening device 104. Responsive to receiving the selection, the measurement component 128 may perform a calibration of the vision screening device 104, in which the measurement component 128 determines whether a pupil is captured in an image using different exposure times (e.g., 6 milliseconds, 12 milliseconds, 18 milliseconds, etc.) by the image capture component 136. In some examples, responsive to receiving the selection, the measurement component 128 may execute a focus process as well, in which the measurement component 128 determines an illumination pattern of the NIR LEDs 138(b) that results in sufficient reflected light being returned to the image capture component 136 from the pupil of the patient 106 to perform various vision screening measurements. Additional details regarding performing calibration and focus actions can be found in U.S. Pat. No. 9,237,846, granted on Jan. 19, 2016, which is incorporated by reference herein in its entirety.

At 504, the measurement component 128 selects and sets an LED pattern of the LED array 138 for an examination protocol. In some examples, the arrangement of the NIR LEDs 138(b) in the LED array allows for different illumination patterns to be presented to the eye(s) of the patient 106. The ocular response of an eye for refractive error may depend upon the illumination pattern selected. For example, less decentered LEDs may offer better resolution for small refractive errors, while more decentered LEDs may extend the range of refractive error that can be detected. By comparing the response of the eye(s) of the patient 106 under different illumination patterns, ambiguities that may arise that are commonly associated with refractive error determinations in conventional systems may be addressed. Additional details regarding illumination patterns used in examination protocols can be found in U.S. Pat. No. 9,237,846, granted on Jan. 19, 2016, which is incorporated by reference herein in its entirety.

At 506, the measurement component 128 acquires a captured image from the image capture component 136. In examples, the image capture component 136 captures an image of the eye(s) of the patient 106 based at least in part on the exposure time(s) determined in the calibration process, and/or the focus settings determined in the focus process, discussed in relation to operation 502. The image capture component 136 may capture the image while the selected LED pattern is displayed by the LED array 138. The image capture component 136 may capture a high-resolution image, such as 752 (horizontal) by 480 (vertical) pixels, although other resolutions are also considered.

At 508, the measurement component 128 determines whether a pupil is detected in the captured image. In cases where the captured image is a high-resolution image as described above, the measurement component 128 may decimate, or sub-sample the high-resolution image to reduce computation time for preliminary isolation of pupil candidates. In an illustrative example, the measurement component 128 may copy every fourth pixel into a sub-sampled array, resulting in an image that is 1/16^{th} of the high-resolution image (e.g., 188/120 pixels). The measurement component 128 may then perform a two-pass procedure on the decimated high-resolution image to enhance pixels likely to be located within the pupil. For instance, the measurement component 128 may determine a region of the pupil in the decimated high-resolution image based on pixels having high intensity and pixels in a non-pupil region having low intensities using a pupil filtering kernel. The threshold component 130 may determine whether the region of the pupil is greater than a threshold size of the decimated high-resolution image (e.g., greater than 30 pixels, greater than 50 pixels, greater than 100 pixels, etc.). If the region of the pupil is greater than the threshold size as determined by the threshold component 130, the measurement component 128 may determine that a pupil is present in the decimated high-resolution image. If the region of the pupil is less than the threshold size as determined by the threshold component 130, the measurement component 128 may determine that a pupil is not present in the decimated high-resolution image. Additional details regarding a pupil filtering kernel can be found in U.S. Pat. No. 9,237,846, granted on Jan. 19, 2016, which is incorporated by reference herein in its entirety.

If the measurement component 128 determines that there is no pupil present in the decimated high-resolution image (e.g., “No” at operation 508), the process 500 may proceed to 510, in which the vision screening device 104 executes a calibration. Similar to the discussion above, the measurement component 128 calibrates the vision screening device 104 by determining whether a pupil is captured in an image using different exposure times (e.g., 6 milliseconds, 12 milliseconds, 18 milliseconds, etc.) by the image capture component 136. Once the calibration is complete, the process 500 returns to operation 504, in which the measurement component 128 selects and sets an LED pattern for an examination protocol, which may be based on the updated calibration in operation 510.

If the measurement component 128 determines that there is pupil present in the decimated high-resolution image (e.g., “Yes” at operation 508), the process 500 may proceed to 512, in which the measurement component 128 determines whether the pupil is acceptable for further analysis. In some examples, the measurement component 128 may determine whether the pupil size(s) are in an accepted size range (e.g., 4 mm-14 mm), whether a change in pupil size(s) between reference images and current images is acceptable (e.g., +/−1 mm between images), whether the inter-pupil distance is in an accepted size range (e.g., 100 mm-300 mm), whether a change in inter-pupil distance between reference images and current images is acceptable (e.g., +/−30 mm between images), whether the gaze direction is acceptable (e.g., within 20 degrees from alignment with the image capture component 136), and the like.

If the measurement component 128 determines that the pupil is not acceptable for further analysis (e.g., “No” at operation 512), the process 500 may return to 510, in which the vision screening device 104 executes a calibration. Similar to the discussion above, the measurement component 128 calibrates the vision screening device 104 by determining whether a pupil is captured in an image using different exposure times (e.g., 6 milliseconds, 12 milliseconds, 18 milliseconds, etc.) by the image capture component 136. Once the calibration is complete, the process 500 returns to operation 504, in which the measurement component 128 selects and sets an LED pattern for an examination protocol, which may be based on the updated calibration in operation 510. Although not explicitly pictured, examples are considered in which the process 500 determines a different focus as a result of the measurement component 128 determining that the pupil is not acceptable for further analysis, alternatively or in addition to performing a calibration.

If the measurement component 128 determines that the pupil is acceptable for further analysis (e.g., “Yes” at operation 512), the process 500 may proceed to 514, in which the measurement component 128 obtains a meridian central profile based on the captured image. For example, the measurement component 128 may determine a diameter of the pupil of the eye as represented in the captured image. As discussed in relation to FIG. 3, the measurement component 128 may also determine an edge width of the pupil along the diameter of the pupil based on a slope between two or more intensity values of pixels along the diameter of the pupil. In some cases, the measurement component 128 may disregard intensity measurements in locations of the captured image determined to be the edge of the pupil when determining a refractive error of the eye. Additionally, in examples, the measurement component 128 may determine the interior of the pupil (e.g., within two edge locations) based on a slope between two or more intensity values in the intensity function being less than a threshold amount, or generally, “flat.” The measurement component 128 may label key points (e.g., k1, k2, k3, etc.) associated with the pupil based on the locations of the edge widths and a center of the flatness profile. For example, the key point k1 may correspond to a first interior of the edge of the pupil, the key point k2 may correspond to a location of the center of the flatness area “F” of the pupil, and the key point k3 may correspond to a second interior edge of the pupil opposite the first interior edge at k1.

At operation 516, the measurement component 128 determines whether the eye(s) of the patient 106 are emmetropic, e.g., have no visual defects (myopia and/or hyperopia) and do not require vision correction. For example, the threshold component 130 may determine a difference between an intensity value at the location of k1 and an intensity value at the location of k3 when each NIR LED at this meridian is on. If the difference is less than a threshold amount and the intensity value at the location of k2 is less than a threshold amount, the measurement component 128 may determine that the eye is emmetropic. If the difference between the intensity value at the location of k1 and the intensity value at the location of k3 is greater than the threshold amount or the intensity value at the location of k2 is greater than a threshold amount, the measurement component 128 may determine that the pupil is not emmetropic.

If the measurement component 128 determines that the eye(s) are emmetropic (e.g., “Yes” at operation 516), the process 500 may proceed to operation 528 in FIG. 6 via “A”, in which the measurement component 128 determines whether the examination protocol calls for an additional meridian of the LED array to be examined. For example, the examination protocol may call for meridians located at 0 degrees, 60 degrees, and 120 degrees to be examined during the vision screening exam to determine if myopia or hyperopia exists along any of these meridians. If the measurement component 128 determines that an additional meridian is called for in the examination protocol that has not yet been examined (e.g., “Yes” at operation 528), the process 500 may return to operation 504 in FIG. 5 via “C” to select and set another LED pattern based on a different meridian.

If the measurement component 128 determines that all of the meridians included in the examination protocol have been examined (e.g., “No” at operation 528), the process 500 may proceed to 530, in the measurement component 128 determines refractive errors in the multiple meridians according to the techniques described in U.S. Pat. No. 9,237,846, granted on Jan. 19, 2016, which is incorporated by reference herein in its entirety. Additionally, at 532, the measurement component 128 generates a vision screening report that includes the refractive errors of the eyes. The measurement component 128 may output the vision screening report to the operator display screen 144 and/or the vision screening system 110.

If the measurement component 128 determines that the eye(s) are not emmetropic (e.g., “No” at operation 516), the process 500 may proceed to 518 in FIG. 6 via “B”, in which the measurement component 128 determines whether myopia is detected the eye(s) of the patient 106. In some examples, the measurement component 128 determines whether myopia is present based on a difference in intensity values at two of the key points determined in the meridian central profile, e.g., k1 and k3. For instance, if the intensity value at k1 is greater than the intensity value at k3, the measurement component 128 may determine that the eye is myopic. If the intensity value at k1 is less than the intensity value at k3, the measurement component 128 may determine that the eye is hyperopic at this meridian.

If the measurement component 128 determines that myopia is present in the eye(s) of the patient 106 (e.g., “Yes” at operation 518), the process 500 may proceed to 520, in which the measurement component 128 adjusts a simulated location of light emitted by the LED array 138 in a first direction. For example, the measurement component 128 may adjust an amount of current delivered to the LED 216 and/or an amount of current delivered to the LED 218 to simulate a change in location of the combined light of the two LEDs. In some cases, the measurement component 128 may control the amount of current delivered to the LED 216 and/or an amount of current delivered to the LED 218 to simulate that the location of the combined light travels outward along a radius associated with a meridian of the row 208 of the LED array 138. The radius along which the measurement component 128 controls the simulated location of the light may be in a direction towards the myopia detected in the captured image.

If the measurement component 128 determines that myopia is not present in the eye(s) of the patient 106 (e.g., “No” at operation 518), the process 500 may proceed to operation 522, in which the measurement component 128 adjusts a simulated location of light emitted by the LED array 138 in a second direction. In examples, the absence of myopia indicates the presence of hyperopia, if refractive error is above a threshold amount as determined in operation 516. Similar to the discussion above, the measurement component 128 may adjust an amount of current delivered to the LED 216 and/or an amount of current delivered to the LED 218 to simulate a change in location of the combined light of the two LEDs. In some cases, the measurement component 128 may control the amount of current delivered to the LED 216 and/or an amount of current delivered to the LED 218 to simulate that the location of the combined light travels outward along a radius associated with a meridian of the row 208 of the LED array 138. The radius along which the measurement component 128 controls the simulated location of the light may be in a direction towards the hyperopia detected in the captured image.

After the simulated location of the light has been adjusted in a first direction or a second direction based on determining whether myopia or hyperopia is present, at 524 the measurement component 128 performs a neutralization eccentricity search to determine an eccentricity at which the refractive error of the eye is neutralized. For example, the measurement component 128 may select location(s) for the simulated source location of the combined light based on a diameter of the pupil of the patient 106. Because different patients have different pupil diameters, a uniform step size between simulated source locations of combined light may cause inaccurate determinations of refractive error. Therefore, the measurement component may, in some examples, cause a first simulated source location to appear at 0.25*d (where d corresponds to the diameter of the pupil of the patient 106 under examination) from the center of the LED array 138 and along a radius of the meridian being evaluated in a direction of the myopia or hyperopia. The measurement component 128 may continue changing the eccentricity by altering an amount of current applied to the LED 216, the LED 218, and/or other LEDs in the LED array until a reflection on the pupil is minimized in an image captured by the image capture component.

At 526, the measurement component 128 determines a meridian refractive error based on the eccentricity at which neutralization was achieved in operation 524. For instance, the measurement component 128 may determine the refractive error based on the correlation with a percentage of eccentricity of the pupil diameter (in millimeters) that is dark at a particular eccentricity distance (in millimeters) from center.

At 528, as described above, the measurement component 128 determines whether the examination protocol calls for an additional meridian of the LED array to be examined. For example, the examination protocol may call for meridians located at 0 degrees, 60 degrees, and 120 degrees to be examined during the vision screening exam to determine if myopia or hyperopia exists along any of these meridians. If the measurement component 128 determines that an additional meridian is called for in the examination protocol that has not yet been examined (e.g., “Yes” at operation 528), the process 500 may return to operation 504 in FIG. 5 via “C” to select and set another LED pattern based on a different meridian.

If the measurement component 128 determines that all of the meridians included in the examination protocol have been examined (e.g., “No” at operation 528), the process 500 may proceed to 530, in which the measurement component determines a refractive error of the eye(s) based at least in part on the refractive error determined in operation 526. In some examples, the measurement component 128 determines refractive errors in the multiple meridians according to the techniques described U.S. Pat. No. 9,237,846, granted on Jan. 19, 2016, which is incorporated by reference herein in its entirety. Additionally, at 532, the measurement component 128 generates a vision screening report that includes the refractive errors of the eyes. The measurement component 128 may output the vision screening report to the operator display screen 144 and/or the vision screening system 110.

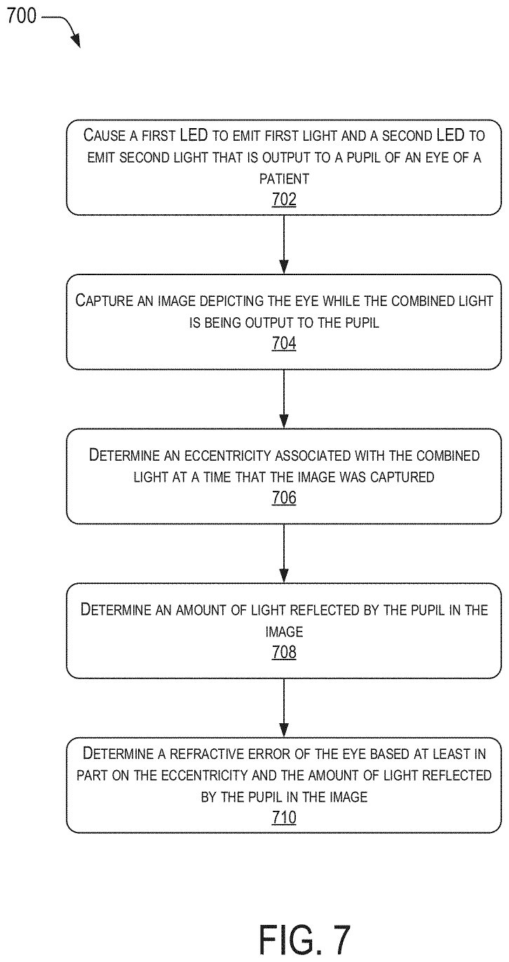

FIG. 7 provides a second flow diagram illustrating an example method 700 of the present disclosure. At 702, the measurement component of the vision screening device 104 causes a first LED 216 to emit first light and a second LED 218 to emit second light, where the combined light of the two LEDs is output to a pupil of an eye of the patient 106. In some examples, the measurement component 128 may cause a current based on the function of current 404 to be applied to the first LED 216 to output the first light, and/or may cause a current based on the function of current 406 to be applied to the second LED 218 to output the second light. The combined light of the first light and the second light may be output to the patient 106 via the diffuser 140, which may act as a blur smooth filter and make the first light and the second light appear to the patient 106 to originate from a same source location.

At 704, the image capture component 136 captures an image depicting the eye while the combined light is being output to the pupil. Additionally, at 706, the measurement component 128 determines an eccentricity associated with the combined light at a time that the image was captured. For example, the measurement component 128 may determine a desired source location for the combined light to appear to the patient 106 based on a diameter of a pupil of the patient, and cause a current to be applied to the LEDs 216 and 218 to emit light to simulate the desired source location for the combined light. The eccentricity may correspond to a distance from a center of the LED array 138 of the simulated source location of the combined light.

At 708, the measurement component 128 determines an amount of light reflected on the pupil in the image. In some examples, the measurement component 128 may use intensity values at key points (e.g., k1, k2, k3 as described in relation to FIG. 3) to determine an amount of light reflected on the pupil in the image. Evaluating light at such key points of the pupil may reduce processing resources of the vision screening device 104 by not determining an amount of light reflected throughout the entire image.

At 710, the measurement component 128 determines a refractive error of the eye based at least in part on the eccentricity and the amount of light reflected on the pupil in the image. In some cases, the measurement component 128 may determine that the eye is emmetropic, if the intensity values at k1 and k2 are within a threshold amount of the intensity value at k3 (e.g., within 0.25 cd) when the light originates from each NIR LED. Alternatively or additionally, the measurement component 128 may determine that the eye is myopic if the intensity value at k1 is greater than the intensity value at k3, or that the eye is hyperopic if the intensity value at k1 is less than the intensity value at k3. Based on a determination that the eye is myopic or hyperopic, the measurement component 128 may alter a current applied to the first LED 216 and/or the second LED 218 to cause the simulated source location of the combined light to move in a direction along a meridian of the LED array 138 until the light reflected on the pupil is minimized (e.g., the intensity value at k1 is within a threshold amount of the intensity value at k3). In some examples, the measurement component 128 may determine whether the eye is myopic and/or hyperopic along other meridians of the LED array 138 as well, to determine an overall refractive error of the eye.

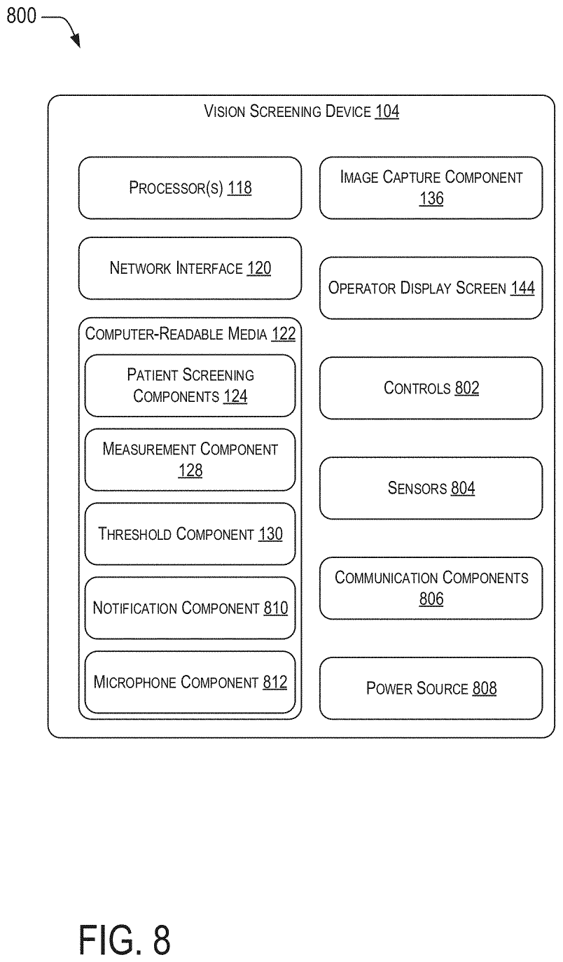

FIG. 8 provides a schematic illustration of an example system 800 of the present disclosure that includes the vision screening device 104. It is understood that any of the components described above with respect to the vision screening device 104 of FIGS. 1 and 2 may be included in the example vision screening device 104 shown schematically in FIG. 8 regardless of whether such components are expressly illustrated in FIG. 8. Additionally, like components between the systems 100 and 200 are illustrated in FIG. 8 using like item numerals. For example, as shown in FIG. 8, the vision screening device 104 may include one or more processors 118 and/or other controller components. The vision screening device 104 may also include the operator display screen 144 and one or more controls 802. In some examples, the controls 802 may receive such inputs from the user 102 during operation of the system 800, and one or more such inputs may comprise a command or a request for the vision screening device 104 to generate, display, provide, and/or otherwise output one or more Snellen charts, characters, or other images included in a visual acuity examination or other vision test. In some cases, the one or more controls 802 may comprise a button, a switch, a trigger, a touchscreen, a keyboard, a microphone, an optical sensor, a video sensor, a camera, and/or other control devices configured to receive touch input, audible commands, visual commands (e.g., hand gestures), and/or other input from the user 102. The controls 802 may generate and/or provide corresponding information to the processor 118 based at least in part on receiving such an input from the user 102. In such examples, the processor 118 may be programmed and/or otherwise configured to perform any of the vision test operations described herein based at least in part on the input, and/or based at least in part on the information received from the controls 802.

The vision screening device 104 shown in FIG. 8 may also include one or more sensors 804, and in some examples, one or more of the sensors 804 may include an accelerometer, a gyroscope, and the like. Alternatively, or additionally, the one or more sensors 804 may comprise one or more the light sensors configured to detect the ambient light intensity around the vision screening device 104. For example, above certain brightness thresholds, the pupils of the patient 106 may constrict to the point where pupil detection is unreliable or impossible. In this instance, the processor 118, in combination with the one or more light sensors, may determine that the ambient light is too bright and the operator display screen 144 may communicate to the user 102 to use a light block, to move to an environment with less ambient light, or in some way adjust the screening environment. Other sensor types are also considered and may be included in the one or more sensors 804, such as proximity sensors, an infrared transceiver unit, an ultrasonic transceiver unit, or another distance measuring component.

Moreover, as will be described in greater detail below, an example vision screening device 104 may include the computer-readable media 122 containing various patient screening components 124, the measurement component 128, and/or the threshold component 130. An example vision screening device 104 may also include the image capture component 136, one or more communication components 806, and/or a power source 808.

In the example shown in FIG. 8, the processor 118 of the vision screening device 104 may comprise one or more controllers, processors, and/or other hardware and/or software components configured to operably control the operator display screen 144, the image capture component 136, the one or more sensors 804, the communication components 806, and/or other components of the vision screening device 104. For instance, the processor 118 shown in FIG. 8 may include a single processing unit (e.g., a single processor) or a number of processing units (e.g., multiple processors), and can include single or multiple computing units or multiple processing cores. The processor 118 shown in FIG. 8 can be implemented as one or more microprocessors, microcomputers, microcontrollers, digital signal processors, central processing units, state machines, logic circuitries, and/or any devices that manipulate signals based on operational instructions. For example, processor 118 shown in FIG. 8 can be one or more hardware processors and/or logic circuits of any suitable type specifically programmed or configured to execute the algorithms, operations, and methods described herein. The processor 118 shown in FIG. 8 can be configured to fetch and execute computer-readable instructions stored in the computer-readable media 122, which can program the processor 118 to perform the functions described herein. Additionally, or alternatively, the processor 118 shown in FIG. 8 can be configured to fetch and execute computer-readable instructions stored in computer-readable media 116 of the vision screening system 110 (FIG. 1).

In any of the examples described herein, the processor 118 shown in FIG. 8 may be configured to receive various information, signals, and/or other inputs from one or more of the controls 802, the sensors 804, the operator display screen 144, the image capture component 136, and/or other components of the vision screening device 104. In some examples, the controls 802 may receive such inputs from the user 102, and one or more such inputs may comprise a command or a request for the vision screening device 104 to generate, display, provide, and/or otherwise output one or more Snellen charts, characters, or other images included in a visual acuity examination or other vision test. One or more such inputs may also comprise a command or a request for the vision screening device 104 to generate, display, provide, and/or otherwise output one or more images, beams of radiation, dynamic stimulus, or other output included in a refractive error examination or other vision test. In examples, the processor 118 shown in FIG. 8 may be operable to cause the visible LEDs 138(a) to generate, display, provide, and/or otherwise output one or more images, beams of radiation, dynamic stimulus, or other output included in a refractive error examination or other vision test.

In some examples, the computer-readable media 122 may include volatile and nonvolatile memory and/or removable and non-removable media implemented in any type of technology for storage of information, such as computer-readable instructions, data structures, program modules, or other data. Such computer-readable media 122 can include, but is not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. The computer-readable media 122 can be a type of computer-readable storage media and/or can be a tangible non-transitory media to the extent that when mentioned, non-transitory computer-readable media exclude media such as energy, carrier signals, electromagnetic waves, and signals per se.

The computer-readable media 122 can be used to store any number of functional components that are executable by the processor(s) 118. In many implementations, these functional components comprise instructions or programs that are executable by the processor(s) 118 and that, when executed, specifically configure the one or more processor(s) 118 to perform the actions described herein and associated with one or more vision screening tests.

The network interface(s) 120 of the vision screening device 104 shown in FIG. 8 may enable wired and/or wireless communications between the vision screening device 104 and one or more components of the vision screening system 110 (FIG. 1), as well as with one or more other remote systems and/or other networked devices. For instance, the interface 120 may include a personal area network component to enable communications over one or more short-range wireless communication channels. Furthermore, the interface 120 may include a wide area network component to enable communication over a wide area network. In any of the examples described herein, the interface 120 may enable communication between the vision screening device 104 and the vision screening system 110 via the network 108 (FIG. 1).

With continued reference to FIG. 8, the computer-readable media 122 may include any number of functional components that are executable by the processor(s) 118. In many implementations, these components comprise instructions or programs that are executable by the processor(s) 118 and that, when executed, specifically configure the one or more processors 118 to perform the actions attributed to the vision screening device 104. For example, as described herein, the patient screening components 124 may be configured to receive, access, store, and/or analyze various data associated with the patient 106 in order to determine patient data for use by the vision screening device 104. For example, the patient screening components 124 may be configured to receive/access patient data indicating demographic information associated with the patient 106. For instance, the patient screening components 124 may be configured to receive patient data entered by the user 102 and indicating the age, ethnicity, gender, name, address, and/or other characteristics of the patient 106 (e.g., patient provided or determined otherwise), as well as a desired vision test to be performed. In examples, the patient screening components 124 may also be configured to receive/access patient data from a database (e.g., the database 126) associated with the vision screening system 110. Still further, in examples, the patient screening components 124 may be configured to receive/access image/video data from the image capture component 136 of the vision screening device 104 and/or any other information from the sensors 804. The patient screening components 124 may be configured to analyze any such information to determine certain characteristics associated with the patient 106, such as visual acuity, refractive error, etc.

Other functional components stored in the computer-readable media 122 may include, among other things, the measurement component 128, the threshold component 130, a machine learning component, and/or any other functional component associated with the operation of the vision screening device 104.

For instance, the computer-readable media 122 may include the measurement component 128 configured to perform functionality as described herein. For example, the measurement component 128 may be configured to receive/access image/video data from the image capture component 136 of the vision screening device 104. The measurement component 128 may also be configured to receive/access sensor data received from any of the sensors 804 described herein. The measurement component 128 may further be configured to analyze such received data to determine one or more measurements associated with the patient 106 throughout the vision test. For example, the measurement component 128 may be configured to analyze the image/video data to determine a location of the patient's pupils and/or lenses, a diameter of the pupils and/or lenses (e.g., indicating expansion or contraction), a motion of the pupils and/or lenses (e.g., indicating a convergence or divergence), a gaze of the patient, and so forth from the data. The measurement component 128 may also be configured to analyze data received from the image capture component 136 and/or the sensors 804 described herein to determine the visual acuity of the patient 106 and/or the refractive error of the patient 106. In examples, the measurement component 128 may be configured to determine one or more measurements associated with the patient 106 during a vision screening exam. For example, the measurement component 128 may be configured to determine the visual acuity, the refractive error, the position of one or both of the patient's pupils, and/or other results of the vision test being performed by the vision screening device 104. In some examples, the measurement component 128 may be configured to analyze the image/video data described herein to determine such results.

For example, the measurement component 128 may be configured to receive/access image/video data from the image capture component 136 of the vision screening device 104 to determine a gaze direction of the patient in response to being displayed the graphical representation. For example, the gaze of the patient 106 may be determined by shining a light, such as an infrared light, in the direction of the patient 106. In response, the cornea of the patient 106 may reflect the light and the reflection may be included, or visible, in the image or video data. The measurement component 128 may utilize the reflection to determine a glint, or straight-line measurement, from the source of the light to the center of the eye (e.g., the origin of the reflection). As such, the measurement component 128 may utilize this information to determine a position, location, and/or motion of the pupil at different points in time while the graphical representation is being displayed. In other examples, the measurement component 128 may utilize the image/video data to determine the position or location of the pupil relative to the outside edges of the eye (e.g., the outline of the eye). The measurement component 128 may utilize the measurements associated with the gaze of the patient to determine one or more locations of the patient's pupils at points in time while being displayed a graphical representation (e.g., position vs. time data points).

In examples, the computer-readable media 122 may also include a threshold component 130. The threshold component 130 may be configured to receive, access, and/or analyze threshold data associated with standard testing results. For example, the threshold component 130 may be configured to access, or receive data from, a third-party database storing testing data and/or measurements, or a range of values indicating a threshold within which testing values should lie, associated with patients having normal vision health with similar testing conditions. For example, for each testing category, standard testing data may be accessed or received by the threshold component 130 and may be utilized for comparison against the measurement data stored by the measurement component 128.

Alternatively, or in addition, the threshold component 130 may be configured to utilize one or more machine learning techniques to determine threshold data associated with each testing category and/or graphical representation. For example, the threshold component 130 may be configured to utilize one or more algorithms and/or trained machine learning models to determine threshold data. For example, the threshold component 130 may execute one or more algorithms (e.g., decision trees, artificial neural networks, association rule learning, or any other machine learning algorithm) to determine the one or more threshold values based on historical vision screening data. In response, the threshold component 130 may be configured to utilize the trained models to determine one or more threshold values and/or standard values for use by the vision screening device 104.

In examples, the computer-readable media 122 may also include a notification component 810. For example, the notification component 810 may be configured to receive and/or access the results of the various vision tests from the measurement component 128, and provide an indication of the results to the user 102 conducting the vision test. For instance, the notification component 810 may be configured to output such results via the operator display screen 144. The notification component 810 may also be configured to provide such results to the vision screening system 110 via the network 108.

In further examples, the computer-readable media 122 may include a microphone component 812. The microphone component 812 may be configured to receive responses spoken by patient 106 and generate audio data associated with the responses. For example, the patient 106 may provide auditory responses as part of the visual acuity test and/or other vision tests described herein. For example, the patient 106 may be asked to read an optotype, such as a letter, shown through the patient viewing window 146 and the microphone component 812 may be configured to receive the patient's responses. In response, the microphone component 812 may be configured to generate audio data associated the responses and/or provide the audio data to the processor 118 shown in FIG. 8. In combination with voice recognition software, the microphone component 812 and/or other functional components of the computer-readable media 122 may decode the responses to generate audio data, and may use the audio data in the various vision tests described herein.

With continued reference to FIG. 8, the image capture component 136 of the vision screening device 104 may be configured to receive and/or access light, image, and/or video data associated with a patient 106 being evaluated during a vision test. In particular, the image capture component 136 may be configured to capture, or generate, image and/or video data during the vision test. For example, as described herein, image data and/or video data may be generated by the image capture component 136 during a vision screening to determine initial patient data, one or more measurements associated with the body and eyes of the patient 106, and the like. In some examples, the image/video data may be transmitted, via the interface(s) 120, to the vision screening system 110 for processing and analysis.

In some examples, the image capture component 136 includes, for example, a complementary metal-oxide semiconductor (CMOS) sensor array, also known as an active pixel sensor (APS), or a charge connected device (CCD) sensor. In some examples, the lens component 134 (not pictured) is supported by the vision screening device 104 and positioned in front of the image capture component 136. In still further examples, the image capture component 136 has a plurality of rows of pixels and a plurality of columns of pixels. For example, the image capture component 136 may include approximately 1280 by 1024 pixels, approximately 640 by 480 pixels, approximately 1500 by 1152 pixels, approximately 2048 by 1536 pixels, and/or approximately 2560 by 1920 pixels. The image capture component 136 may be capable of capturing approximately 25 frames per second (fps), approximately 30 fps, approximately 35 fps, approximately 40 fps, approximately 50 fps, approximately 75 fps, approximately 100 fps, approximately 150 fps, approximately 200 fps, approximately 225 fps, and/or approximately 250 fps. Note that the above pixel values and frames per second are exemplary, and other values may be greater or less than the examples described herein.

In examples, the image capture component 136 may include photodiodes having a light-receiving surface and have substantially uniform length and width. During exposure, the photodiodes convert the incident light to a charge. The image capture component 136 may be operated as a global shutter. For example, substantially all of the photodiodes may be exposed simultaneously and for substantially identical lengths of time. Alternatively, the image capture component 136 may be used with a rolling shutter mechanism, in which exposures move as a wave from one side of an image to the other. Other mechanisms are possible to operate the image capture component 136 in yet other examples. The image capture component 136 may also be configured to capture digital images. The digital images can be captured in various formats, such as JPEG, BITMAP, TIFF, etc.

The communication components 806 of the example vision screening device 104 shown in FIG. 8 may be configured to connect to external databases (e.g., the database 126) to receive, access, and/or send screening data using wireless connections. Wireless connections can include cellular network connections and connections made using protocols such as 802.11a, b, g, and/or ac. In other examples, a wireless connection can be accomplished directly between the vision screening device 104 and an external display using one or more wired or wireless protocols, such as Bluetooth, Wi-Fi Direct, radio-frequency identification (RFID), or Zigbee. Other configurations are possible. The communication of data to an external database can enable report printing or further assessment of the patient's visual test data. For example, data collected and corresponding test results may be wirelessly transmitted and stored in a remote database accessible by authorized medical professionals.

Further, it is understood that the power source 808 may comprise any removable, rechargeable, and/or other power source known in the art and configured to store electrical power. The power source 808 may comprise one or more rechargeable batteries configured to selectively provide electrical current to the one or more components of the vision screening device 104 during use. For instance, the power source 808 may comprise one or more sealed lead acid batteries, lithium ion batteries, nickel cadmium batteries, nickel-metal hydride batteries, or other types of batteries configured to provide sufficient power to operator display screen 144, the one or more processors 118, the image capture component 136, and/or other components of the visions screening device 104 during multiple vision tests.

Based at least on the description herein, it is understood that the systems and methods of the present disclosure may be used to assist in performing one or more visual acuity tests, refractive error tests, or other vision tests. For example, components of the systems described herein may be configured to determine the refractive error based on eccentricity of one or more beams relative to a pupil of a person's eye that achieved neutralization of the beams. In some examples, rather than mechanically moving the light sources used to determine refractive error, the system may adjust the intensities of the light sources to simulate movement. Thus, the system described herein may reduce an overall size of the vision screening device and/or a cost of the vision screening device by removing the need for mechanical components that were required in previous systems to utilize eccentricity to determine refractive error.

The foregoing is merely illustrative of the principles of this disclosure and various modifications can be made by those skilled in the art without departing from the scope of this disclosure. The above described examples are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

What is claimed is:

1. A system comprising:
a first light-emitting diode (LED);
a second LED;
an image capture component;
one or more processors; and
one or more computer-readable media storing instructions that, when executed by the one or more processors, perform operations comprising:
causing the first LED to emit first light and the second LED to emit second light to form a combined light that is output to a pupil of an eye of a patient;
causing the image capture component to capture an image depicting the eye while the combined light is being output to the pupil;
determining an eccentricity associated with the combined light at a time that the image was captured, the eccentricity corresponding to a simulated source location of the combined light caused by combining the first light and the second light;
determining an amount of light reflected on the pupil in the image; and
determining a refractive error of the eye based at least in part on the eccentricity and the amount of light reflected on the pupil in the image.

2. The system of claim 1, the operations further comprising:
determining the amount of light reflected on the pupil in the image is less than a threshold amount of light; and
determining that the refractive error is neutralized at the eccentricity based at least in part on the amount of light reflected on the pupil being less than the threshold amount.

3. The system of claim 1, further comprising a diffuser, wherein the combined light is output to the pupil via the diffuser.

4. The system of claim 3, wherein the diffuser diffuses the first light and the second light as a low-pass filter to form the combined light.

5. The system of claim 1, wherein the combined light is a first combined light, the image is a first image, the eccentricity is a first eccentricity, the time is a first time, and the amount of light is a first amount of light, the operations further comprising:
altering an amount of current applied to one or more of the first LED or the second LED such that one or more of the first light or the second light increases intensity to form a second combined light;
causing the image capture component to capture a second image depicting the eye while the second combined light is being output to the pupil;
determining a second eccentricity associated with the second combined light at a second time that the second image was captured; and
determining a second amount of light reflected on the pupil in the second image,
wherein determining the refractive error is further based on the second eccentricity and the second amount of light reflected on the pupil in the second image.

6. The system of claim 5, wherein the simulated source location is a first simulated source location, and wherein the second eccentricity corresponds to a second simulated source location of the second combined light caused by combining the first light and the second light with the increased intensity.

7. The system of claim 6, wherein the second simulated source location is located external to the first simulated source location relative to a center of the image capture component, and along a radius of a circle centered on the image capture component.

8. The system of claim 6, the operations further comprising determining a diameter of the pupil, and wherein a distance between the first simulated source location and the second simulated source location is based at least in part on the diameter of the pupil.

9. A method comprising:
causing a first light-emitting diode (LED) of a vision screening device to emit first light and a second LED of the vision screening device to emit second light to form a combined light that is output to a pupil of an eye of a patient;
capturing, by an image capture component of the vision screening device, an image depicting the eye while the combined light is being output to the pupil;
determining an eccentricity associated with the combined light at a time that the image was captured, the eccentricity corresponding to a simulated source location of the combined light caused by combining the first light and the second light;
determining an amount of light reflected on the pupil in the image; and
determining a refractive error of the eye based at least in part on the eccentricity and the amount of light reflected on the pupil in the image.

10. The method of claim 9, further comprising:
determining the amount of light reflected on the pupil in the image is less than a threshold amount of light; and
determining that the refractive error is neutralized at the eccentricity based at least in part on the amount of light reflected on the pupil being less than the threshold amount.

11. The method of claim 9, wherein the combined light is output to the pupil via a diffuser of the vision screening device, and wherein the diffuser diffuses the first light and the second light as a low-pass filter to form the combined light.

12. The method of claim 9, wherein the combined light is a first combined light, the image is a first image, the eccentricity is a first eccentricity, the time is a first time, and the amount of light is a first amount of light, the method further comprising:
altering an amount of current applied to one or more of the first LED or the second LED such that one or more of the first light or the second light increases intensity to form a second combined light;
capturing, by the image capture component, a second image depicting the eye while the second combined light is being output to the pupil;
determining a second eccentricity associated with the second combined light at a second time that the second image was captured; and
determining a second amount of light reflected on the pupil in the second image,
wherein determining the refractive error is further based on the second eccentricity and the second amount of light reflected on the pupil in the second image.

13. The method of claim 12, wherein the simulated source location is a first simulated source location, and wherein the second eccentricity corresponds to a second simulated source location of the second combined light caused by combining the first light and the second light with the increased intensity.

14. The method of claim 13, wherein the second simulated source location is located external to the first simulated source location relative to a center of the image capture component, and along a radius of a circle centered on the image capture component.

15. The method of claim 13, further comprising determining a diameter of the pupil, and wherein a distance between the first simulated source location and the second simulated source location is based at least in part on the diameter of the pupil.

16. One or more computer-readable media storing instructions that, when executed by one or more processors, cause the one or more processors to perform operations comprising:
causing a first light-emitting diode (LED) of a vision screening device to emit first light and a second LED of the vision screening device to emit second light to form a combined light that is output to a pupil of an eye of a patient;
capturing, by an image capture component of the vision screening device, an image depicting the eye while the combined light is being output to the pupil;
determining an eccentricity associated with the combined light at a time that the image was captured, the eccentricity corresponding to a simulated source location of the combined light caused by combining the first light and the second light;
determining an amount of light reflected on the pupil in the image; and
determining a refractive error of the eye based at least in part on the eccentricity and the amount of light reflected on the pupil in the image.

17. The one or more computer-readable media of claim 16, wherein the combined light is a first combined light, the image is a first image, the eccentricity is a first eccentricity, the time is a first time, and the amount of light is a first amount of light, the operations further comprising:
altering an amount of current applied to one or more of the first LED or the second LED such that one or more of the first light or the second light increases intensity to form a second combined light;
capturing, by the image capture component, a second image depicting the eye while the second combined light is being output to the pupil;
determining a second eccentricity associated with the second combined light at a second time that the second image was captured; and
determining a second amount of light reflected on the pupil in the second image, wherein determining the refractive error is further based on the second eccentricity and the second amount of light reflected on the pupil in the second image.

18. The one or more computer-readable media of claim 17, wherein the simulated source location is a first simulated source location, and wherein the second eccentricity corresponds to a second simulated source location of the second combined light caused by combining the first light and the second light with the increased intensity.

19. The one or more computer-readable media of claim 18, wherein the second simulated source location is located external to the first simulated source location relative to a center of the image capture component, and along a radius of a circle centered on the image capture component.

20. The one or more computer-readable media of claim 16, the operations further comprising determining a diameter of the pupil, and wherein a distance between the first simulated source location and the second simulated source location is based at least in part on the diameter of the pupil.
